(19) United States
(12) Patent Application Publication
(10) Pub. No.: US 2007/0280895 A1
(43) Pub. Date: Dec. 6, 2007

(54) Coated particles and sunscreen and cosmetic products containing same

(76) Inventors: Alan W. Weimer, Niwot, CO (US); Steven M. George, Boulder, CO (US)

Correspondence Address: GARY C. COHN, PLLC, 1147 NORTH FOURTH STREET, UNIT 6E, PHILADELPHIA, PA, 19123, US

(21) Appl. No.: 11/801,124

(22) Filed: May 8, 2007

Related U.S. Application Data
(60) Provisional application No. 60/810,362, filed on Jun. 2, 2006.
(60) Provisional application No. 60/843,445, filed on Sep. 8, 2006.

(51) Int. Cl. A61K 8/29; A61K 8/27

(52) U.S. Cl. 424/59; 977/926

(57) ABSTRACT

Particles have an ultrathin, conformal coating are made using atomic layer deposition methods. The base particles include ceramic and metallic materials. The coatings can also be ceramic or metal materials that can be deposited in a binary reaction sequence. The coated particles are useful as fillers for electronic packaging applications, for making ceramic or cermet parts, as supported catalysts, as well as other applications.

### CROSS-REFERENCE TO RELATED APPLICATION

This application claims benefit of U.S. Provisional Application No. 60/810,362, filed Jun. 2, 2006, and of U.S. Provisional Application No. 60/843,445, filed Sep. 8, 2006.

### BACKGROUND OF THE INVENTION

This invention relates to particulate materials that are useful as sunscreen agents, and to topical sunscreen or cosmetic compositions containing sunscreen agents.

Topical sunscreen products protect the skin from the effects of harmful ultraviolet (UV) radiation by preventing the transmission of that radiation through absorption and scattering processes. A variety of sunscreen agents have been used in such products. One type of sunscreen agent that is widely used is a particulate metal oxide. Titanium dioxide and zinc oxide particles are most commonly used. Sometimes combinations of these are used.

The difficulty with using metal oxides as the active sunscreen agent is that it is difficult to formulate a product that is transparent and yet has a high sunscreen protection factor (SPF). When the metal oxide is present in a concentration that is high enough to produce a high SPF, the formulation usually becomes white and opaque. Transparency is quite an important attribute in sunscreen formulations for aesthetic reasons, as users do not want a white residue to appear on their skin when they use the product. Similarly, many cosmetic products such as foundations are formulated to produce a specific color or tone, but cannot be done so when the metal oxide content is too high.

A number of approaches have been tried to resolve this problem. One approach is to supplement the metal oxide with an organic sunscreen agent. The desired SPF can be achieved in this way due to the combined effect of the metal oxide and organic sunscreen agents. This allows a sunscreen to be formulated using lower levels of the metal oxide particles, and in that way reduces the amount of opacity of the product. The organic sunscreen agents are often susceptible to photodegradation and/or chemical degradation by peroxides or other superoxides, hydroxyl compounds and/or free radicals. Unfortunately, metal oxides often produce oxidants or free radicals of this sort when exposed to UV light. As a result, the organic sunscreen agent can rapidly degrade when the product is exposed to sunlight, and the product SPF value drops rapidly. It has been attempted to passivate the surface of the metal oxide particles to overcome this problem. See, e.g., U.S. Published Patent Application 2005/0255057, which describes an attempt to coat metal oxide particles to avoid the problem of degrading organic sunscreen agents. In EP 803 550, it has been suggested to dope the metal oxide particles with another metal or a reduced zinc oxide in order to reduce their tendency to generate reactive species which degrade the organic sunscreen agents. Doping can dilute the efficiency of the metal oxide.

An approach to solving the whiteness problem is to use very small metal oxide particles. Examples of this approach are described, for example, in U.S. Published Patent Applications 2003/061795, 2005/0255057 and WO 2005/002538. As a rule of thumb, particles that are of the order of one-tenth the wavelength of visible light or less would be expected to be transparent to visible light, if adequately dispersed into a carrier. Particles in the primary size range of 20-50 nm, therefore, would be expected to scatter visible light very poorly, yet retain sufficient ability to scatter the shorter UV light. Several factors have made this difficult in practice.

### SUMMARY OF THE INVENTION

In one aspect, this invention is a material in the form of particles having an average primary particle size in the range of 20 to 1000 nm, wherein the particles comprise a core and at least one titanium dioxide, zinc oxide or cerium oxide shell layer having a thickness of from 1 to 1000 Angstroms. The invention is also a topical sunscreen composition or cosmetic composition comprising such particles dispersed in a dermatologically acceptable carrier, the composition exhibiting a Sun Protection Factor of at least +8.

In another aspect, this invention is a material in the form of particles having an average primary particle size in the range of from 5-70 nm, the particles comprising a core and at least one external zinc oxide layer having a thickness of from 1 to 100 angstroms. The invention is also a topical sunscreen composition or cosmetic composition comprising such particles dispersed in a dermatologically acceptable carrier, the composition exhibiting a Sun Protection Factor of at least +8.

In yet another aspect, this invention is a material in the form of particles having a titanium oxide or zinc oxide surface that is covered with a passivating, non-photocatalytically active layer, for example aluminum oxide or silicon dioxide, having a thickness of from 1 to 500 angstroms. The invention is also a topical sunscreen composition or cosmetic composition comprising such particles dispersed in a dermatologically acceptable carrier, the composition exhibiting a Sun Protection Factor of at least +8.

### DETAILED DESCRIPTION OF THE INVENTION

Particles according to one aspect of the invention include a core that has one or more layers of titanium dioxide, zinc oxide or cerium oxide. The particles have an average primary particle size of from 10 to 1000 nm. Particle sizes for purposes of this invention are volume average particle sizes, with the longest dimension of a particle being considered to represent the primary particle size. As very fine particles tend to agglomerate due to Van der Waals or electrostatic forces, particles sizes herein are those of the “primary”, or unagglomerated particles. The particles of the invention preferably have an average size of from 20 to 500 nm, more preferably from 20 to 200 nm, even more preferably from 20 to 150 nm, still more preferably from 20-100 nm and especially from 20 to 70 nm.

The particles comprise a core and at least one shell layer that is different in composition than the core. At least one shell layer is composed of titanium dioxide, zinc oxide or cerium oxide. Preferably, at least one shell layer is composed of titanium dioxide or zinc oxide, and most preferably the particle contains at least one shell layer of zinc oxide. Except for one or more optional passivating layers as described further below, a shell layer forms the outermost surface of the particle.

Each shell layer has a thickness of from 1 to 1000 angstroms (0.1 to 100 nm). A preferred shell layer thickness is from 10 to 500 angstroms (1 to 50 nm). A more preferred shell layer thickness is from 10 to 100 angstroms (1 to 10 nm). A still more preferred shell layer thickness is from 10 to 75 anstroms (1 to 7.5 nm).

The core can be made of any material that can be provided as a particulate of the required primary size, regardless of geometry, and upon which the shell layer(s) can be formed. The composition of the core is different from that of at least one shell layer. Inorganic materials are generally preferred, as well as carbon particles. The core preferably is a material that has hydroxyl or other functional groups on it surface, as those particles are susceptible to being coating via an atomic layer deposition process as described more fully below. Particularly preferred core materials include silica, carbon, titanium dioxide, zinc oxide, boron nitride, aluminum nitride, and the like, as well as metals such as iron, nickel or aluminum. Silicon dioxide cores are of particular interest as they are readily available in the desired particle size, have a low refractive index, are not subject to rapid oxidation, and are readily susceptible to the application of shell layers via an atomic layer deposition process.

The core advantageously has a particle size of from 5 to 998 nanometers. A preferred core has a primary particle size of from 5 to 498 nanometers. A more preferred core has a particle size of from 10 to 198 nm. An even more preferred core has a particle size of from 10 to 148 nm, and a still more preferred particle size is from 15 to 98 nm. An especially preferred core has a particle size of from 15 to 68 nm.

In certain embodiments of the invention, the particle includes a core and a titanium dioxide shell layer. The titanium dioxide shell layer in this embodiment preferably has a thickness of from 10 to 500 angstroms, more preferably from 25 to 250 angstroms and especially from 25 to 100 angstroms. A silica core is of particular interest in such embodiments. A preferred particle of this embodiment has an average primary particle size of 20 to 50 nm and a titanium dioxide shell layer from 10 to 100 angstroms, especially 20-75 angstroms, thick.

In other embodiments of the invention, the particle includes a core and a zinc oxide shell layer. The zinc oxide shell layer in this embodiment preferably has a thickness of from 10 to 500 angstroms, more preferably from 25 to 250 angstroms and especially from 50 to 100 angstroms. A silicon dioxide core or titanium dioxide core are of particular interest in such embodiments. A preferred particle of this embodiment has an average primary particle size of 20 to 50 nm and a zinc oxide shell layer from 10 to 100 angstroms, especially 20-75 angstroms, thick.

In still other embodiments of the invention, the particle includes a core and a cerium oxide shell layer. The cerium oxide shell layer in this embodiment preferably has a thickness of from 10 to 500 angstroms, more preferably from 25 to 250 angstroms and especially from 50 to 100 angstroms. A silicon dioxide core is of particular interest in such embodiments.

In still other embodiments of the invention, the particle includes a core, at least one titanium dioxide or cerium oxide layer, and at least one zinc oxide layer that is exterior to the titanium dioxide or cerium oxide layer. The zinc oxide layer preferably resides directly on top of a one titanium dioxide or cerium oxide layer, which preferably resides directly on the core. At least one zinc oxide layer more preferably resides on the surface of the particle, except for an optional passivating layer as described for fully below. The titanium dioxide or cerium oxide layer and the zinc oxide shell layer, preferably each have a thickness of from 10 to 500 angstroms, more preferably from 25 to 250 angstroms and especially from 50 to 100 angstroms. A silicon dioxide core is of particular interest in such embodiments.

Another aspect of the invention is a material in the form of particles having a titanium oxide or zinc oxide surface that is covered with a passivating aluminum oxide layer having a thickness of from 10 to 500 angstroms. In this aspect, the particle may be made entirely of titanium oxide or zinc oxide. Alternatively, the particle may be a core-shell particle such as those just described, which has a titanium dioxide or zinc oxide surface layer covered with the passivating aluminum oxide layer. In particular, the particle of this aspect may have:

A) A core, a titanium dioxide or zinc oxide layer atop the core, and an aluminum oxide layer on top of the titanium dioxide or zinc oxide layer. In this embodiment, the core is preferably silica, the titanium dioxide or zinc oxide layer is preferably from about 10 to 500 angstroms, more preferably from 25 to 250 angstroms and especially from 50 to 100 angstroms in thickness, and the aluminum oxide passivating layer is from about 10-500, especially from 10 to 100 angstroms in thickness. A preferred particle of this embodiment has an average particle size of 20 to 50 nm and a titanium dioxide or zinc oxide shell layer from 10 to 100 angstroms, especially 20-75 angstroms, thick, and an aluminum oxide passivating layer from 10 to 100 angstroms thick.

B) A core, a titanium dioxide layer atop the core, a zinc oxide layer atop the titanium dioxide layer, and an aluminum oxide layer on top of the zinc oxide layer. In this embodiment, the core is preferably silicon dioxide, the titanium dioxide and zinc oxide layers each are preferably from about 10 to 500 angstroms, more preferably from 25 to 250 angstroms and especially from 50 to 100 angstroms in thickness, and the aluminum oxide passivating layer is from about 10 to 500, especially from 10 to 100 angstroms in thickness. A preferred particle of this embodiment has an average particle size of 20 to 50 nm, titanium dioxide and zinc oxide shell layers from 10 to 100 angstroms thick, especially 20-75 angstroms thick, and the aluminum oxide layer is from 10 to 100 angstroms thick.

The various oxide shell layers are conveniently provided using chemical vapor deposition and/or atomic layer deposition processes. In chemical vapor deposition (CVD), the core particles are introduced into a reaction zone, and gaseous precursor compounds are then introduced into the reaction zone. The precursors react in the gas phase to form droplets of the coating material. These droplets deposit on the surface of the core particles to form the desired coating.

It is so difficult to form very thin, uniform films using CVD processes as to be impractical. Atomic layer deposition (ALD) processes are therefore generally preferred, because ALD has proven successful, with a variety of precursor materials, to deposit very thin, uniform coatings. ALD is distinguished from CVD in the manner in which the precursors are introduced into the reaction zone. In ALD processes, the precursor materials are introduced sequentially, with removal of excess precursor materials between successive introductions of reactants. In this manner, reactions occur at the particle surface, rather than in the gaseous phase. Each sequence of reactions deposits an atomic mono-layer of material onto the particle surface. Atomic layer controlled growth techniques permit the formation of deposits of 0.015 to 0.3 nm in thickness per reaction cycle, depending on coating material and process conditions, and thus provide a means of extremely fine control over deposit thickness. In these techniques, the deposits are formed in a series of two or more self-limited reactions, which in most instances can be repeated to sequentially deposit additional layers of the deposited material until a desired thickness is achieved. This causes very uniform films to be deposited. Very thin films can be produced in this manner. The process also permits very precise control over the film thickness, by controlling the number of times the reaction sequence is repeated.

Suitable methods for applying coatings to particles via the ALD process are described in U.S. Pat. Nos. 6,913,827; 6,713,177; and 6,613,383. The particles to be coated are contacted with the reagent gases while retained in a reaction vessel that permits gases to flow past the particles and for temperatures to be controlled as needed. A convenient method for applying the ultrathin, conformal coating to the base particles is to form a fluidized bed of the particles, and then pass the various reagents in turn through the fluidized bed under reaction conditions. In addition, the reactions can be conducted in a rotating cylindrical vessel or a rotating tube.

In most instances, the first of the ALD reactions will involve some functional group on the surface of the particle, such as an M-H, M-O—H or M-N—H group, where M represents an atom of a metal or semi-metal. If necessary, the surface of the particle can be treated to introduce such functional groups. Depending on the particular base particle, techniques such as water plasma treatment, ozone treatment, ammonia treatment and hydrogen treatment are among the useful methods of introducing functional groups.

It is preferred to treat the particles before initiating the reaction sequence to remove volatile materials that may be adsorbed onto the surface. This is readily done by exposing the particles to elevated temperatures and/or vacuum. Also, in some instances a precursor reaction may be done to introduce desirable functional groups onto the surface of the particle, as described before.

The coated particulate is preferably non-agglomerated after the inorganic material is deposited. By “non-agglomerated”, it means that the particles do not form significant amounts of agglomerates during the process of coating the substrate particles with the inorganic material. Particles are considered to be non-agglomerated if (a) the average particle size does not increase more than about 5%, preferably not more than about 2%, more preferably not more than about 1% (apart from particle size increases attributable to the coating itself) as a result of depositing the coating, or (b) if no more than 2 weight %, preferably no more than 1 weight % of the particles become agglomerated during the process of depositing the inorganic material.

Zinc oxide, cerium oxide or titanium dioxide, can be prepared on particles having surface hydroxyl or amine (M-N—H) groups using a binary (AB) reaction sequence as follows. The asterisk (*) indicates the atom that resides at the surface of the particle or coating, and Z represents oxygen or nitrogen. M¹ represents a metal or semi-metal atom, e.g. zinc, titanium or cerium, and X is a displaceable nucleophilic group. The reactions shown below are not balanced, and are only intended to show the reactions at the surface of the particles (i.e., not inter- or intralayer reactions).
M-Z-H*+M¹Xₙ→M-Z-M¹X*+HX (A1)
M-Z-M¹X*+H₂O→M-Z-M¹OH*+HX (B1)
In reaction A1, reagent M¹Xₙ reacts with one or more M*-Z-H groups on the surface of the particle to create a new surface group having the form -M¹-X. M¹ is bonded to the particle through one or more Z atoms. The -M¹-X group represents a site that can react with water in reaction B1 to regenerate one or more hydroxyl groups. The water can in some cases be replace with other oxidizing agents such as hydrogen peroxide (H₂O₂) or ozone (O₃). In some cases, the M₁Xₙ compound is thermally degradable to the desired layer material and in such cases the second reagent can be omitted. The hydroxyl groups formed in reaction B1 can serve as functional groups through which reactions A1 and B1 can be repeated, each time adding a new layer of M¹ atoms. Hydroxyl groups can be eliminated as water, forming M¹-O-M¹ bonds within or between layers. This condensation reaction can be promoted if desired by, for example, annealing at elevated temperatures and/or reduced pressures. The subscript ‘n’ in reaction A1 is not necessarily a whole number, as processing conditions, as well as post treatments, may be used to preferentially alter the stoichiometric ratio to enhance the effectiveness of the invention.

Suitable M₁X compounds for applying a zinc oxide film are diethyl zinc and dimethyl zinc. Suitable M₁X compound for applying a titanium dioxide film are titanium tetraisopropoxide and titanium tetraethoxide. Suitable M₁X compounds for applying a cerium oxide film are cerium tetramethylheptanedionate and cerium trimethylheptanedionate phenanthroline. In some cases, the corresponding metal halides can be used.

A reaction scheme for the deposition of TiO₂ is described in Tsapatsis et al. (1991) Ind. Eng. Chem. Res. 30:2152-2159 and Lin et al., (1992), AlChE Journal 38:445-454, both incorporated herein by reference.

In addition, catalyzed binary reaction techniques such as described in copending application Ser. No. 08/942,522 entitled “Method of Growing Films on Substrates at Room Temperatures Using Catalyzed Binary Reaction Sequence Chemistry”, incorporated by reference, are suitable for depositing inorganic materials, especially oxide, nitride or sulfide coatings, most preferably oxide coatings. Reactions of this type can be represented as follows:
M-F₁+C₁→M-F₁—C₁ (A4a)
M-F₁—C₁+F₂-M¹-F₂→M-M¹-F₂+F₁—F₂+C₁ (A4b)
M-M¹-F₂+C₂→M-M¹-F₁—C₂ (B4a)
M-M¹-F₁—C₂+F₁-M-F₁→M-M¹-M-F₁+F₁—F₂+C₂ (B4b)
C₁ and C₂ represent catalysts for the A4b and B4b reactions, and may be the same or different. F₁ and F₂ represent functional groups, and M and M¹ are as defined before, and can be the same or different. Reactions A4a and A4b together constitute the first part of a binary reaction sequence, and reactions B4a and B4b together constitute the second half of the binary reaction sequence.

Except for the catalyzed reaction scheme described above, the binary reactions are generally performed at elevated temperatures, preferably from about 300-1000K. Between reactions, the particles are subjected to conditions sufficient to remove reaction products and unreacted reagents. This can be done, for example, by subjecting the particles to a high vacuum, such as about 10⁻⁵ Torr or less, after each reaction step. Another method of accomplishing this, which is more readily applicable for industrial application, is to sweep the particles with an inert purge gas between the reaction steps. This purge gas can also act as a fluidizing medium for the particles and as a carrier for the reagents.

Several techniques are useful for monitoring the progress of the reaction. For example, vibrational spectroscopic studies can be performed on high surface area silica powders using transmission Fourier transform infrared techniques. The deposited inorganic materials can be examined using in situ spectroscopic ellipsometry. Atomic force microscopy studies can be used to characterize the roughness of the coating relative to that of the surface of the substrate. X-ray photoelectron spectroscopy and x-ray diffraction can be used to do depth-profiling and ascertain the crystallographic structure of the coating. Mass Spectroscopy, or Residual Gas Analysis, downstream of the reactor can be utilized in situ to monitor gaseous products, then gaseous reactants, that pass through the reaction chamber while dosing each precursor. Once the reaction product is no longer generated, the reaction is complete and any additional precursor dosed into the system is wasted. A convenient method for applying the ultrathin deposits of inorganic material to the base particles is to form a fluidized bed of the particles, and then pass the various reactants in turn through the fluidized bed under appropriate process conditions. Methods of fluidizing particulate materials are well known, and generally include supporting the particles on a porous plate or screen. A fluidizing gas is passed upwardly through the plate or screen, lifting the particles somewhat and expanding the volume of the bed. With appropriate expansion, the particles behave much as a fluid. Fluid (gaseous or liquid) reagents can be introduced into the bed for reaction with the surface of the particles. In this invention, the fluidizing gas also can act as an inert purge gas for removing unreacted reagents and volatile or gaseous reaction products.

In addition, the reactions can be conducted in a rotating cylindrical vessel or a rotating tube. A rotating reactor comprises a hollow tube that contains the particulate. The reactor is held at an angle to the horizontal, and the particulate passes through the tube through gravitational action. The reactor angle determines the flow rate of the particulate through the reactor. The reactor is rotated in order to distribute individual particles evenly and expose all particles to the reactants. The reactor design permits the substrate particles to flow in a near plug-flow condition, and is particularly suitable for continuous operations. The reactants are introduced individually and sequentially through the reactor, preferentially countercurrent to the direction of the substrate particles.

The coated particles are useful as active UV protection agents in sunscreens and other topical cosmetic formulations. Examples of topical cosmetic formulations include, for example, ointments, creams, face powders, blushes, eye shadows, bronzing products, lip products such as lip gloss, lip paint, lip powder, lip liners, lip pencils, lip plumping and lipsticks, concealers, emulsions or multiple emulsions, nail care products, leave-on-hair compositions, deodorants, anti-perspirants, and the like. The products may be in the form of a cream, ointment, paste, aerosol, powder, stick or other suitable form.

The formulated product typically contains one or more other ingredients, which are selected in conjunction with the particular application and particular attributes they impart to the particular product. The additional ingredients may include, for example, one or more surfactants, emulsifiers, fats, thickeners and stabilizers, waxes, electrolytes, moisturizers, water, water-soluble polymers, film-formers, thickeners, antimicrobials and other preservatives, pH adjusting agents, emollients, antioxidants, free radical scavengers, fragrances, organic sunscreen agents, solvents, photostabilizing agents, vitamins, tanning agents, coloring agents driers or other materials. Examples of such materials are described in a wide variety of references, including, for example, US Published Patent Application Nos. 2005/0255057, 2003/0219391 and 2003/0161795.

Examples of suitable emulsifiers include polyglycerol esters, sorbitan esters and partially esterified glycerides. Examples of fats include glycerides of animal or plant origin. Useful stabilizers include metal salts of fatty acids such as magnesium aluminum or zinc stearate. Examples of thickeners and stabilizers include magnesium aluminum silicate, sodium aluminum silicate, colloidal silica, fumed silica, sodium stearate, steareth 20 methacrylate copolymer, PEG150/decyl alcohol/SMDI copolymer, PEG150/stearyl alcohol/SMDI copolymer, polyethylene, hydrophobic silica, metal stearates such as zinc stearate and the like. Suitable waxes include pariffin, beeswax and microcrystalline waxes, optionally used in conjunction with hydrophilic waxes.

Examples of suitable moisturizers include urea, glycolic acid and its salts, lactid acid and its salts, aloe vera, sorbitol, glycerol, butylene glycol, hexylene glycol and other polyyhydric alcohols, polyethylene glycol, sugar and its derivatives, starch and its derivatives, hyaluronic acid and its salts, urea, guanidine and the like. Suitable water-soluble polymers and/or film-formers include xanthan gum, cellulose derivatives, acrylic acid polymers and copolymers, carbomers, PVP, alginates, guar gum, chitosan, vinylpyrrolidone/vinyl acetate coplymers, quaternary cellulose derivatives and the like.

Examples of suitable antimicrobials and other preservatives include formaldehyde, p-hydroxybenzoate and sorbic acid. A wide range of organic or inorganic acids and bases can be added to adjust pH. Suitable emollients include hydrocarbon oils such as paraffin oil and mineral oil, vegetable oils such as sunflower oil, apricot oil, jojobal oil, shea butter, silicone oil, fatty acid esters such as isopropyl palmitate, isopropyl myristate, isopropyl neo-entanoate, cetearyl octanoate, C₁₂₋₁₅ alkyl benzoate, cetyl palmitate, octyl palmitate and the like.

Suitable antioxidants include amino acids such as glycine, histidine, tyrosine and tryptophan, as well as derivatives thereof, peptides such as D,L-carnosine, D-carnosine, L-carnosine and derivatives thereof, retinoids such as retinol, retinal and/or retinoic acid and its esters, carotenoids, carotines and derivatives thereof, α-lipoic acid and derivatives thereof, aurothioglucose, propylthiouracil and other thiols, dilauryl thiodipropionate, distearyl thiodipropionate, thiodipropionic acid and derivatives thereof, sulfoximine compounds and metal chelating agents such as α-hydroxy acids, humic acid, bile acid, bile extracts, bilirubin, biliverdin, EDTA, EGTA, unsaturated fatty acids and derivatives thereof, folic acid and derivatives thereof, 2-aminopropionic acid, diacetic acid, flavonoids, polyphenols, catechins, ubiquinone, ubiquinol, vitamin C, tocopherols and derivatives such as vitamin E, coniferyl benzoate, rutinic acid and derivatives thereof, ferulic acid and derivatives thereof, butylhydroxytoluene, butylhydroxyanisole, nordihydroguaiacic acid, nordihydroguaiaretic acid, trihydroxybutylrophenone, uric acid and derivatives thereof, mannose and its derivatives, zinc oxide, zinc sulfate, selenium compounds, stilbene and its derivatives, and the like, as further described in US Published Patent Application 2005/0255057.

Suitable organic sunscreen agents include 2-ethylhexyl-p-methoxycinnamate, isoamyl-p-methoxycinnimate, 2-ethoxyethyl p-methoxycinnamate, 2-ethylhexyl N,N-dimethyl-p-aminobenzoate, 4-aminobenzoic acid, 2-phenyl-benzimidazole-5-sulfonic acid and its salts, homosalate, oxybenzone, 2-ethylhexyl salicylate, 3-(4′-methylbenzylidene)-1-camphor, benzophenone-2, benzophenone-4, benzophenone-5, dioxybenzone, menthyl anthranilate, octocrylene, octyl triazone, triethanolamine salicylate, PEG 25 PABA, avobenzone and mixtures thereof.

The particles of the invention are also useful as additives in paints, coatings, organic polymers and other materials in which it is desired to provide some protection from ultraviolet light. In such instances, the presence of the particles of the invention can retard, reduce or even prevent discoloration that often appears in paints, coatings and polymers due to the effects of incident ultraviolet radiation. Organic polymers that contain aromatic groups tend to be particularly prone to such discoloration, as do paints and coatings that contain such polymers. Examples of polymers that contain aromatic groups include, for example, poly(vinyl aromatic) polymers such as polystyrene and polystyrene copolymers, impact-modified polystyrene polymers such as ABS and SAN resins; aromatic polyesters such as those based on terephthalic acid or isophthalic acid (such as PET resins), polyurethanes and polyureas based on MDI or TDI; aromatic polycarbonates such as those based on a bisphenol compound; aromatic epoxy resins such as those based on a bisphenol compound; and the like.

The following examples are provided to illustrate the invention, but are not intended to limit its scope. All parts and percentages are by weight unless otherwise indicated.

### EXAMPLE 1

### A Atomic Layer Deposition (ALD) of Zinc Oxide on Titania Nanoparticles

A stainless steel rotary fluidized bed reactor is used to deposit a zinc oxide coating on 30 nanometer titanium dioxide particles. The reactor includes a porous metal cylinder for holding the particles, which is located within a vacuum system. The cylinder has an inside diameter of 7.5 cm, a length of 20 cm and an average pore size of 20 microns.

20 grams of the TiO₂ particles (P-25, from DeGussa) are placed in the stainless steel porous metal cylinder. This fills the porous metal cylinder to approximately 10% of the total volume. The system was pumped to a base pressure of 30 mTorr, and the reactor is then rotated at 90 rpm. The reactor is heated to 180° C. prior to the introduction of reactants, and held at that temperature through the reaction process. Reactants are preheated to 60° C. to ensure that they had a vapor pressure higher than the dose pressure. First, diethylzinc (DEZ) is introduced to the reactor to a pressure of 65 Torr, and allowed to react statically for 60 seconds. The system is then evacuated to base pressure and the chamber is filled with nitrogen to a pressure of 30 Torr. This nitrogen purge is held statically for 5 seconds, and the reactor is then evacuated to base pressure again for 120 seconds. Water vapor is then introduced to the reactor to a pressure of 65 Torr and allowed to react statically for 60 seconds, after which the chamber is again evacuated to base pressure. A second nitrogen purge step is then performed as before. This sequence of reactions is repeated 30 times. The coated particles have a zinc oxide coating approximately 4.5 nanometers thick. The coated particles (Particle Example 1) contain about 42% by weight zinc oxide and 58% by weight of titanium dioxide.

XPS analysis of the surface of the resulting particles indicates that the surface is coated with zinc oxide, as evidenced by the appearance of an oxide shifted Zn 2p^{3/2} peak in the spectrum at a binding energy of 1021.7 eV.

49.5 parts of the particles are thoroughly mixed into 50.5 parts of mineral oil to form Sunscreen Formulation 1-1. Sunscreen Protection Factor is measured for this formulation using a Labsphere UV1000-S Transmittance Analyzer across the UVA (320-400 nm) and UVB (290-320 nm) spectra. The Labsphere UV1000-S contains an integrating sphere that combines all forward scattered light into the value for transmittance; backscattered and absorbed light are calculated as one value as the difference between light emitted from the unit (directly into the sample) and light collected within the integrating sphere. SPF testing is done using VITRO-SKIM™ (IMS Corporation) as the test vehicle. The formulation is applied at a surface concentration of 2 mg/cm², using an FDA and COLIPA approved measurement procedure. Results of this tested are as reported in Table 1.

For comparison (Comparative Sample A1), 28.7 parts of the uncoated titanium dioxide base particles are dispersed in 71.3 parts of mineral oil. This blend contains the same loading of titania as does Sunscreen Formulation 1-1. SPF is measured as for Sunscreen Formulation 1; results of this testing are as reported in Table 1. Separately, a blend (Comparative Sample B1) of 20.8 parts of nano-sized ZnO particles (from Sigma Aldrich, 20 m²/g surface area, mean diameter 60 nm) and 79.2 parts of mineral oil is prepared. This blend contains the same loading of zinc oxide as does Sunscreen Formulation 1-1. Again, SPF is measured as for Sunscreen Formulation 1 with results of this testing being as reported in Table 1.

TABLE 1

| Sunscreen Formulation or | TiO₂, | ZnO, | Mineral Oil, | |
|---|---|---|---|---|
| Comparative Sample No. | wt-% | wt-% | wt-% | SPF |
| 1-1 | 28.7 | 20.8 | 50.5 | 162 |
| A1* | 28.7 | 0 | 71.3 | 111 |
| B1* | 0 | 20.8 | 79.2 | 28 |

*Not an example of the invention

The coated nanoparticles of the invention provide a much higher sunscreen protection factor on these tests.

Blends containing 2% by weight of nanoparticles in 98% by weight mineral oil are prepared. The UVA and UVB transmittance of the blends is then evaluated. The identification of the particles and results of this testing are as reported in Table 2.

TABLE 2

| Sunscreen Formulation | | | | | |
|---|---|---|---|---|---|
| or Comparative Sample | Particle | | | UVA | UVB |
| No. | Description | TiO₂, wt-% | ZnO, wt-% | trans., % | trans., % |
| 1-2 | Ex. 1, ZnO | 1.1 | 0.9 | 56.4 | 45.4 |
| | on TiO₂ | | | | |
| A2* | TiO₂ base | 2.0 | 0 | 71.2 | 63.1 |
| | particles | | | | |
| B2* | ZnO nano- | 0 | 2.0 | 66.0 | 64.0 |
| | particles | | | | |
| C* | Mixed TiO₂ | 1.1 | 0.9 | 64.5 | 59.0 |
| | and ZnO | | | | |
| | nanoparticles | | | | |

*Not an example of the invention.

These results show that, at equal total particle loadings, the zinc oxide-coated titanium dioxide particles of the invention provide superior UV protection.

### EXAMPLE 2

### Synthesis of Improved UV Blocking Nanoparticles with Improved Non-whitening Characteristics

A 45 Angstrom thick ZnO film is placed on 30 nanometer average diameter Degussa P-25 TiO₂ particles in a rotary fluidized bed reactor, in the manner described in Example 1. The resulting particles have an average diameter of approximately 35-40 nm, and contain about 42% zinc oxide. 2 parts of the resulting particles are blended into 98 parts of mineral oil to form Sunscreen Formulation 2. Sunscreen Formulation 2 is applied to Vitro-Skin test vehicle at a loading of 2 mg/cm2 and tested for UVA and UVB transmission as described in Example 1. The coated test vehicle is also inspected visually for signs of opacity. The applied formulation is found to be clear.

For comparison, a blend of 2% of the 60 nm zinc oxide particles described in Example 1 is formed in 98% mineral oil (Comparative Sample D). Comparative Sample D forms a somewhat opaque coating on the test vehicle.

### EXAMPLES 3-5

### Atomic Layer Deposition (ALD) of Titania on Silica Particles

A low-pressure fluidized bed reactor is used to deposit titania onto silica base particles. The SiO₂ particles are commercially available from Presperse Inc. under the trade name Cosmo-55. Primary Cosmo-55 particles are spherical and 550 nm micron in diameter. They form agglomerates in the 10-30 micron size range. The total surface area of the Cosmo-55 particles is approximately 5.6 m²/g.

The reactor apparatus is divided into a dose zone, a reactor zone and a pump zone, which are arranged in that order. A pneumatically actuated diaphragm valve separates the dose and reactor zones, and a manual gate valve separates the reactor and pump zones. Continuous rough vacuum is maintained in the pump zone by a rotary vane vacuum pump. A bypass line connects the dose zone to the pump zone, divided by a normally closed pneumatic diaphragm valve. Both pneumatic valves are controlled by the same solid-state relay. The dose zone is comprised of two precursor lines and a carrier gas line. Each precursor line connects to a canister that holds the respective reagent

The reactor zone consists of a stainless steel fluidized bed reactor mounted within an externally controlled clamshell-type furnace. The fluidized bed is 5.1 cm in diameter and 91.4 cm long. A porous stainless steel distributor plate (10 micron pore size) with a thickness of approximately 1.5 mm is used to support the contents of the reactor. The top of the reactor expands to accommodate a 6 inch flange sealed with a copper gasket. A low pressure sensor (1-10 Torr) is affixed to a port located on top of the 6 inch flange such that the pressure above the fluid bed may be monitored.

15.05 grams of the Cosmo-55 particles are charged to the reactor. Fluidization is maintained with nitrogen carrier gas (e.g. N₂), the flow of which is controlled by a Mass Flow Controller. The reactor is heated to a temperature of 300° C. and maintained at that temperature throughout the course of the reaction cycles. The reactants (titanium tetraisopropoxide (TTIP) and water) are charged to the reactor according to the following protocol:

| Step | Description | Time (s) |
|---|---|---|
| 1 | Quick Purge | 2 |
| 2 | TTIP Dose | 90 |
| 3 | N2 Flush | 90 |
| 4 | Quick Purge | 2 |
| 5 | N2 Flush | 90 |
| 6 | Quick Purge | 2 |
| 7 | H2O Dose | 120 |
| 8 | N2 Flush | 120 |
| 9 | Quick Purge | 2 |
| 10 | N2 Flush | 120 |

This pattern is repeated until coating approximately 15 Angstroms thick is added to the surface of the silica particles to form Example 3. At this coating thickness, TEM analysis was inconclusive as the resolution was too low to detect a layer of this expected thickness relative to the primary particle size.

Example 4 is made in the same manner, except 50.10 g of SiO₂ particles are coated, and the reaction cycle is repeated enough times to produce a TiO₂ coating approximately 37.5 angstroms thick. Example 5 is made by coating 30.59 g of the Example 4 particles in the same manner, until the TiO₂ is 50 angstroms thick.

Portions of each of particle Examples 3, 4 and 5 are separately blended with mineral oil to form blends containing 5%, 15% and 25% of the particles. The UVA and UVB transmission of the resulting blends is measured in the manner described in Example 1. Results are as reported in Table 4.

TABLE 4

| | Particles | | Transmission Results, % | |
|---|---|---|---|---|
| | Ex. No. | TiO₂ Thickness | UVA | UVB |
| 5% Loading | 3 | 15 Angstroms | 100 | 99.6 |
| Results | 4 | 37.5 Angstroms | 99.5 | 94.9 |
| | 5 | 50 Angstroms | 98.1 | 93.7 |
| 15% Loading | 3 | 15 Angstroms | 98.2 | 97.1 |
| Results | 4 | 37.5 Angstroms | 98.5 | 98.6 |
| | 5 | 50 Angstroms | 98.1 | 96.3 |
| 25% Loading | 3 | 15 Angstroms | 95.4 | 92.5 |
| Results | 4 | 37.5 Angstroms | 90.8 | 84.4 |
| | 5 | 50 Angstroms | 91.3 | 87.4 |

These results show that ALD methods can be used to place an active UV blocking TiO₂ film on inert substrate particles and activate the particles to absorb UV rays. The amount of absorption increases with the thickness of the TiO₂ and with the loading of the activated particles in a formulation.

### EXAMPLES 6-8

### Atomic Layer Deposition (ALD) of Zinc Oxide on Titania Coated Silica

Examples 6-8 are made in the general manner described in Examples 3-5, except that diethyl zinc is used in place of TTIP, and zinc oxide coatings are instead formed on the particles. Particle Example 6 has a zinc oxide coating approximately 45 angstroms. Particle Example 7 has a zinc oxide coating of approximately 135 angstroms. Particle Example 8 has a zinc oxide coating of approximately 225 Angstroms.

The resulting particles are separately blended with mineral oil to form blends containing 5%, 15% and 25% of the particles. UVA and UVB transmission is evaluated for these blends as described in Example 1, with results as indicated in Table 5.

TABLE 5

| | Particles | | Transmission Results, % | |
|---|---|---|---|---|
| | Ex. No. | ZnO Thickness | UVA | UVB |
| 5% Loading | 3 | 45 Angstroms | 97.4 | 92.9 |
| Results | 4 | 135 Angstroms | 84.0 | 78.6 |
| | 5 | 225 Angstroms | 78.5 | 75.0 |
| 15% Loading | 3 | 45 Angstroms | 93.3 | 91.4 |
| Results | 4 | 135 Angstroms | 59.3 | 47.2 |
| | 5 | 225 Angstroms | 52.2 | 42.0 |
| 25% Loading | 3 | 45 Angstroms | 90.8 | 87.4 |
| Results | 4 | 135 Angstroms | 43.4 | 32.2 |
| | 5 | 225 Angstroms | 43.1 | 37.2 |

These results show that ALD methods can be used to place an active UV blocking ZnO film on inert substrate particles and activate the particles to absorb UV rays. The amount of absorption increases with the thickness of the ZnO coating and with the loading of the activated particles in a formulation.

### EXAMPLE 9

Particle Example 9 is made using a general process similar to that described in Examples 3-8, but using a scaled stainless steel fluid bed reactor. This reactor includes a porous metal frit at the and a porous metal cylindrical filter at the top. It has an internal diameter of 1.37″ and a height of 16″. 25 grams of 60 rim ZnO particles (Z-Cote, from BASF) are placed in the reactor, which is subsequently heated to 300° C. The particle bed is fluidized using a nitrogen flow rate of 60 sccm. TTIP is dosed by diverting the nitrogen flow through a bubbler containing the precursor, which is heated to approximately 75° C. Dionized water, stored at room temperature, is directly dosed into the nitrogen flow via its vapor pressure, which is higher than operating pressure. Reactants are charged to the reactor according to the following protocol (1 cycle):

| Step | Description | Time (s) |
|---|---|---|
| 1 | TTIP Dose | 180 |
| 2 | N2 Flush | 180 |
| 3 | Water Dose | 180 |
| 4 | N2 Flush | 300 |

A TiO₂ shell having a thickness of 14 Angstroms is deposited using 41 consecutive TTIP-H₂O cycles.

The resulting coated particles are analyzed using Inductively Coupled Plasma-Atomic Emission Spectroscopy (ICP-AES). They contain 92.46% by weight ZnO and 7.54% by weight titania. The titania layer is 13.6 angstroms thick, which correlates to a growth rate of 0.33 Angstroms/reaction cycle.

### EXAMPLE 10

Particle example 10 is made in the same general manner described in Example 9, this time using hydrogen peroxide as the oxidant. 21.80 grams of the ZnO particles are placed in the reactor apparatus described in Examples 3-8, which is subsequently heated to 100° C. TTIP is fed in as described in Example 9 and hydrogen peroxide (H₂O₂, 50% in H₂O) is fed in the same manner as described for the water addition in Example 9. Reactants are charged into the reactor according to the following protocol (1 cycle).

| Step | Description | Time (s) |
|---|---|---|
| 1 | TTIP Dose | 150 |
| 2 | N2 Flush | 180 |
| 3 | H₂O₂ Dose | 120 |
| 4 | N2 Flush | 240 |

This reaction cycle is repeated 75 times. The resulting coated particles are analyzed using Inductively Coupled Plasma-Atomic Emission Spectroscopy (ICP-AES). They contain 93.1% by weight ZnO and 6.9% by weight titania. The titania layer is 12.36 angstroms thick, which correlates to a growth rate of 0.11 Angstroms/reaction cycle.

2.0, 5.0 and 8.0 parts of the resulting particles are blended into 98, 95 and 92 parts of a 50:50 butylene glycol:water (BGW) mixture, respectively to form Sunscreen Formulations 10-1, 10-2 and 10-3. Each sunscreen formulation is applied to VITRO-SKIN test vehicle at a loading of 2 mg/cm² and tested for UVA and UVB transmittance as described in Example 1. Results are as indicated in Table 6. The coated test vehicles are also inspected visually for signs of opacity. All applied formulations are found to have less of a whitening behavior than identically loaded mixtures of uncoated TiO₂ and ZnO substrate particles at equivalent weight percent loadings. The data in FIG. 9 also demonstrate the positive correlation between TiO₂ loading increase and UVB shielding, and the positive correlation between ZnO loading increase and UVA shielding.

TABLE 6

| Particle Description | | | | | | | |
|---|---|---|---|---|---|---|---|
| Sunscreen | Active | | | Transmittance (%) | | UVA/UVB | |
| Formulation | % | TiO2 % | ZnO % | UVA | UVB | Ratio | SPF |
| 10-1 | 2.00 | 0.14 | 1.86 | 76.62 | 73.01 | 0.85 | 1.4 |
| 10-2 | 5.00 | 0.35 | 4.65 | 45.56 | 39.25 | 0.87 | 2.5 |
| 10-3 | 8.00 | 0.55 | 7.45 | 9.98 | 3.33 | 0.83 | 29.6 |

### EXAMPLE 11

Particle Example 11 are prepared using the same general procedure described in Example 9, this time depositing a 12 Angstom alumina layer on the ZnO particles followed by addition of a 15 Angstrom titania layer. The alumina layer is deposited at about 180° C. using 10 cycles of the following protocol:

| Step | Description | Time (s) |
|---|---|---|
| 1 | TMA Dose | 180 |
| 2 | N2 Flush | 150 |
| 3 | Water Dose | 120 |
| 4 | N2 Flush | 300 |

The reactor temperature is then raised to 300° C., and the titania layer is deposited using 25 cycles of the following protocol:

| Step | Description | Time (s) |
|---|---|---|
| 1 | TiCl₄ Dose | 240 |
| 2 | N2 Flush | 120 |
| 3 | Water Dose | 120 |
| 4 | N2 Flush | 300 |

The resulting coated particles are analyzed using Inductively Coupled Plasma-Atomic Emission Sspectroscopy (ICP-AES). They contain 87.58% by weight ZnO, 4.61% by weight alumina and 7.54% by weight titania. The alumina layer is 11.7 angstroms thick, which correlates to a growth rate of 1.17 Angstroms/reaction cycle. The titania layer is 14.85 angstroms thick, which correlates to a growth rate of 0.59 Angstroms/reaction cycle.

### EXAMPLE 12

### Synthesis of Improved UV Blocking Nanoparticles in Butylene Glycol:Water Mixture for Enhanced Sunscreen Formulation

A 20 Angstrom thick ZnO shell is deposited onto 30 nanometer average diameter TiO₂ particles (Degussa P-25 particles) in the general manner described in Examples 6-8. The resulting particles have an average diameter of approximately 30-35 nm and contain about 13% zinc oxide. 2.0, 5.0 and 8.0 parts of the resulting particles are blended into 98, 95 and 92 parts of a 50:50 butylene glycol:water (BGW) mixture, respectively to form Sunscreen Formulations 12-1, 12-2 and 12-3. The resulting formulations are evaluated as described in Example 10, with results as indicated in Table 7.

TABLE 7

| Particle Description | | | | | | | |
|---|---|---|---|---|---|---|---|
| Sunscreen | Active | | | Transmittance (%) | | UVA/UVB | |
| Formulation | % | TiO2 % | ZnO % | UVA | UVB | Ratio | SPF |
| 12-1 | 2.00 | 1.74 | 0.26 | 45.69 | 32.66 | 0.72 | 3.3 |
| 12-2 | 5.00 | 4.34 | 0.66 | 25.02 | 12.47 | 0.71 | 8.1 |
| 12-3 | 8.00 | 6.95 | 1.05 | 10.62 | 2.77 | 0.71 | 37.0 |

### EXAMPLES 13-15

### Atomic Layer Deposition (ALD) of Titania and Zinc Oxide Nanolaminates on Silica Particles with an Alumina Capping Layer

A low-pressure fluidized bed reactor similar to that described in Examples 3-5 is used to deposit titania and zinc oxide shells onto silica base particles. The silica base particles are commercially available (Sigma Aldrich), spherical particles that have a primary particle diameter of 100 nm. 12.50 grams of 100 nm SiO₂ particles are charged to the reactor. The reactor is heated to a temperature of 100° C. and maintained at that temperature throughout the course of the entire process. 180 TTIP-H₂O₂ cycles are used to deposit a film thickness of 3 nm, in a manner similar to that described in Example 10. A ZnO layer of similar thickness is then deposited using 35 DEZ-H₂O cycles to form Particle Example 13. This 3 nm ZnO layer on 3 nm TiO₂ layer is considered one multilayer (ML) stack and is designated N=1. Particle Examples 14 and 15 correspond to N=5 and 10, respectively. The particles are analyzed as described in Example 11, with results as indicated in Table 8.

An alumina passivating layer is then deposited over each of Particle Examples 13-15 using the general process described in Example 11.

2.0 weight percent mixtures in BGW are created using each of Particle Examples 13-15. The mixture are analyzed using VITRO-SKIN, as in the method described in Example 1. Results are as indicated in Table 8.

TABLE 8

| Particle Description | | | | | Transmittance (%) | |
|---|---|---|---|---|---|---|
| Ex. No. | N | SiO₂ % | TiO₂ % | ZnO % | UVA | UVB |
| 13 | 1 | 50 | 20 | 30 | 65.46 | 48.31 |
| 14 | 5 | 33 | 27 | 40 | 31.91 | 26.36 |
| 15 | 10 | 20 | 32 | 48 | 21.72 | 14.49 |

These results show that ALD methods can be used to place an active UV blocking TiO₂/ZnO ML stacks on inert substrate particles and activate the particles to absorb UV rays. The amount of absorption increases with the thickness and loading of TiO₂ and ZnO ALD films. The alumina capping layer (not included in mass percent calculations) protects these optically active cosmetics substrate particles from photocatalytically degrading contacting surfaces, for example human skin.

### EXAMPLES 16-18

### Atomic Layer Deposition (ALD) of Alumina and Zinc Oxide Nanolaminates on Silica Particles with an Alumina Capping Layer

12.50 grams of 100 nm SiO₂ particles are charged to a reactor as described in Examples 3-5. The reactor is heated to a temperature of 100° C. and maintained at that temperature throughout the course of the entire process. A zinc oxide film with a thickness of 5 nm is deposited using 50 DEZ-H₂O reaction cycles, in the manner described in Examples 13-15. A conformal alumina layer of similar thickness is then deposited atop the zinc oxide film using 50 TMA-H₂O reaction cycles as described in Example 11, to produce Particle Example 16. This 5 nm Al₂O₃ layer on 5 nm ZnO layer is considered one multilayer (ML) stack and is designated N=1. Examples 17 and 18 correspond to N=5 and 10, respectively. The particles are analyzed as described in Example 11, with results as indicated in Table 9.

2.0 weight percent mixtures in BGW are created using each of Particle Examples 16-18. The mixtures are analyzed using VITRO-SKIN, as in the method described in Example 1. Results are a s indicated in Table 9.

TABLE 9

| Particle Description | | | | | Transmittance (%) | |
|---|---|---|---|---|---|---|
| Ex. No. | N | SiO₂ % | TiO₂ % | ZnO % | UVA | UVB |
| 23 | 1 | 38 | 43 | 19 | 51.16 | 43.31 |
| 24 | 5 | 17 | 56 | 27 | 18.34 | 12.66 |
| 25 | 10 | 20 | 48 | 32 | 8.98 | 3.06 |

The results show that ALD methods can be used to place an active UV blocking Al₂O₃/ZnO ML stacks on inert substrate particles and activate the particles to absorb UV rays. The alternating high and low refractive index of the coating materials activates the particles to efficiently scatter UV rays. The amount of absorption increases with the thickness and loading of the ZnO ALD films, whereas Al₂O₃ films are not significantly active in the UV regime. The amount of scattering directly increases with N, the number of concentric ML stacks. The alumina capping layer, which is inherently included in the mass percent here due to Al₂O₃ being the second of two components in each ML stack. Similar to previous examples the capping layer protects these optically active cosmetics substrate particles from phocatalytically degrading contacting surfaces, for example human skin.

What is claimed is:

1. A material in the form of particles having an average particle size in the range of 5 to 1000 nm, wherein the particles comprise a core and at least one titanium dioxide, zinc oxide or cerium oxide shell layer having a thickness of from 1 to 1000 Angstroms.

2. A topical sunscreen composition or cosmetic composition comprising the material of claim 1 dispersed in a dermatologically acceptable carrier, the composition exhibiting a Sun Protection Factor of at least +8.

3. A material in the form of particles having an average particle size in the range of from 20-50 nm, the particles comprising a core and at least one external zinc oxide layer fabricated using atomic layer deposition having a thickness of from 1 to 100 angstroms.

4. A topical sunscreen composition or cosmetic composition comprising the material of claim 3 dispersed in a dermatologically acceptable carrier, the composition exhibiting a Sun Protection Factor of at least +8.

5. A material in the form of particles having a titanium oxide or zinc oxide surface that is covered with a passivating aluminum oxide or silicon dioxide layer fabricated using atomic layer deposition having a thickness of from 1 to 500 angstroms.

6. A topical sunscreen composition or cosmetic composition the material of claim 5 dispersed in a dermatologically acceptable carrier, the composition exhibiting a Sun Protection Factor of at least +8.
